(19) United States
(12) Patent Application Publication
(10) Pub. No.: US 2012/0251449 A1
(43) Pub. Date: Oct. 4, 2012

(54) ALDH: A COMPOUND FOR CANCER STEM CELL IMAGING

(76) Inventors: Vijaya Raj Kuniyil Kulangara, Bangalore (IN); Alan Cuthbertson, Oslo (NO); Peter Iveson, Amersham (GB); Chitralekha Rangaswamy, Bangalore (IN); Veena Rao, Bangalore (IN); Rajiv Bhalla, Amersham (GB)

(21) Appl. No.: 13/510,398

(22) PCT Filed: Dec. 22, 2010
(86) PCT No.: PCT/US10/61697

§ 371 Date: May 17, 2012

(30) Foreign Application Priority Data
Dec. 22, 2009 (IN) 2685/DEL/2009

(51) Int. Cl. A61K 51/04; C07C 47/55; A61P 35/00; C07C 223/06; C07C 223/04; C07C 47/353

(52) U.S. Cl. 424/1.89; 564/1; 568/420; 424/1.85; 564/462; 564/458; 564/426; 564/442; 568/425; 568/439; 568/445

(57) ABSTRACT

The present invention claims fluoro- & iodo-containing aldehydes as ALDH substrates for use as diagnostic imaging agents or as therapeutic agents. The aldehydes are both directly and indirectly attached to an aromatic or a straight chain ring. ALDH activity was monitored either by the formation of acid-product or consumption of aldehyde substrates.

### FIELD OF THE INVENTION

The present invention allows for the investigation for finding substrates for aldehyde dehydrogenase with characteristics to allow imaging of Human Liver Aldehyde Dehydrogenases (ALDH) in vivo. The compounds tested were iodinated or fluorinated aldehydes.

### BACKGROUND OF THE INVENTION

Across the world, there have been many reported cases of cancer recurrence after initial treatment using surgery, chemotherapy or radiotherapy. It is believed that some cancerous cells that are left behind may cause recurrence. These cells may remain dormant for a long period of time, but eventually could continue to multiply, resulting in the relapse of cancer.

There is a strong unmet need to be able to predict recurrence of the disease and the ability to detect cells that are left behind and cause relapse could give valuable information as far as therapy monitoring and predicting recurrence of cancer is concerned. Recently the stem cell model of cancer has emerged based on the principle that a sub-population of tumour initiating cells are present in the tumour which are distinct from the bulk cells of the tumour. The model predicts that eradication of the bulk of the tumour cells by chemotherapy or radiotherapy will at best result in temporary remission if cancer stem cells are left behind following treatment. It is also known that these stem cell-like populations are more resistant to many of the alkylating agents used in standard chemotherapy regimes. For example, clinical studies have shown the benefit of purging samples with 4-hydroperoxycyclophosphamide (4-HC) before autologous bone marrow transplantation (ABMT) which removes committed progenitor cells but leaves the stem cell population largely intact. In addition, breast cancer studies have demonstrated correlation between ALDH expression in tumour tissue and poor clinical outcome and have also suggested ALDH as a marker of malignant mammary stem cells. Kinetics and Specificity of Human Liver Aldehyde Dehydrogenases (ALDH) toward aliphatic, aromatic, and fused polycyclic aldehydes are reported in the literature. Also Aldefluor and Dansylaminoacetaldehyde are two substrates for ALDH that are reported in the literature where they have been used for the in vitro separation of stem cells using flow cytometry techniques. The primary purpose of the invention is to present substrates for aldehyde dehydrogenase with characteristics that allow imaging of ALDH in vivo. Specifically, the compounds of interest were iodinated or fluorinated aldehydes.

### DETAILED DESCRIPTION OF THE INVENTION

The iodinated and fluorinated aldehydes disclosed herein can be used as cancer stem cell targets. These aldehydes can be used to enable detection or diagnosis of breast, colon, liver, myeloma, or AML cancers. Specific use of compounds of formulas (I) and (II) are used to detect and separate cancer stem cells by in vitro ALDEFLUOR® assay. The ALDEFLUOR® fluorescent reagent system offers an approach to the identification, evaluation, and isolation of stem and progenitor cells based on their expression of the enzyme, aldehyde dehydorgenase (ALDH), rather than cell surface phenotype. ALDEFLUOR® is used to detect stem and progenitor cells in multiple lineages includes hematopoietic, mammary, mesenchymal, endothelial, and neural. It is adaptable for use with other species and cell types, including cancer stem cells. It is used to identify only viable cells with an intact cellular membrane and is suitable for cryopreserved or fresh samples.

The principle of designing the disclosed radionuclide aldehyde substrates for PET and SPECT imaging involves an uptake of these substrates by stem cells whereby these substrates are processed by ALDH to give a negatively charged dye. The dye accumulates in the stem cell and the cells are sorted by flow cytometry. Flow cytometry is a technique for counting and examining microscopic particles, such as cells, by suspending them in a stream of fluid and passing them by an electronic detection apparatus. It allows simultaneous multiparametric analysis of the physical and/or chemical characteristics of up to thousands of particles per second.

One embodiment of the present invention depicts a compound for tumor stem cell imaging of ALDH in vivo wherein the compound is a radionuclide of formula I comprising:

A—Ar—NH—Cₙ—CH═O (I)

wherein A is ¹⁸F or ¹²³I, Ar is a 6- or 8-carbon member-ring or a 6-, 8-, 10-, or 12-member aliphatic chain ring, or a fused polycyclic ring and n is 1-4.

Yet another embodiment of the present invention depicts a compound for tumor stem cell imaging of ALDH in vivo wherein the compound is a radionuclide of formula II comprising:

A—Ar—CH═O (II)

wherein A is ¹⁸F or ¹²³I, Ar is a 6- or 8-carbon member-ring or a 6-, 8-, 10-, or 12-member aliphatic chain ring or a fused polycyclic ring.

Still a further embodiment of the invention depicts a pharmaceutical composition comprising an effective amount of a compound of formula (I) or a salt thereof, together with one or more pharmaceutically acceptable adjuvants, excipients or diluents for use in enhancing image contrast in in vivo imaging or for treatment of a disease.

Yet another embodiment depicts a pharmaceutical composition comprising an effective amount of a compound of formula (II) or a salt thereof, together with one or more pharmaceutically acceptable adjuvants, excipients or diluents for use in enhancing image contrast in in vivo imaging or for treatment of a disease.

Still another use of the present invention depicts a compound as claimed in claim 1 or claim 2 in the preparation of a contrast medium for use in a method of diagnosis involving administering said contrast medium to a human or animal body and generating an image of at least part of said body.

And yet another embodiment of the invention depicts a method of generating images of a human or animal body involving administering a compound of formula I to said body, and generating an image of at least a part of said body to which said contrast agent has distributed, characterised in that said compound comprises a compound as claimed in claim 1 or claim 2.

Still another embodiment of the invention depicts a method of generating enhanced images of a human or animal body comprising a compound as claimed in claim 1 or claim 2, which method comprises generating an image of at least part of said body.

Yet another embodiment of the invention depicts a method of monitoring the effect of treatment of a human or animal body with a drug to combat a condition associated with cancer, said method involving administering to said body a compound or composition as claimed in claim 1 and detecting the uptake of said compound or composition by cell receptors, said administration and detection optionally but preferably being effected repeatedly, e.g. before, during and after treatment with said compound or composition.

Still another embodiment of the invention depicts a method of treating cancer or a related disease in a human or animal body which comprises the administration of an effective amount of a compound or composition as claimed claim 1 or claim 2.

Another embodiment of the invention depicts a use of a compound as claimed in claim 1 or claim 2 for the manufacture of a medicament for the therapeutic or prophylactic treatment of cancer or a related disease in a human or animal.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

The following are references cited for use herein: 1) Tannishtha Reya, et al., Nature, Vol 414, November 2001, 105-111 2) R. J. Hones et al., Blood, 1195, 1995 85: 2742-2746; 3) Anatole A. Klyosov, Biochemistry 1996, 35, 4457-4467; 4) Piero Dalerba et al., Annu. Rev. Med. 2007.58:267-284.

What is claimed is:

1. A compound for tumor stem cell imaging of ALDH in vivo wherein the compound is a radionuclide of formula I comprising:
A—Ar—NH—Cₙ—CH═O (I)
wherein A is ¹⁸F or ¹²³I, Ar is a 6- or 8-carbon member-ring or a 6-, 8-, 10-, or 12-member aliphatic chain ring, or a fused polycyclic ring and n is 1-4.

2. A compound for tumor stem cell imaging of ALDH in vivo wherein the compound is a radionuclide of formula II comprising:
A—Ar—CH═O (II)
wherein A is ¹⁸F or ¹²³I, Ar is a 6- or 8-carbon member-ring or a 6-, 8-, 10-, or 12-member aliphatic chain ring, or a fused polycyclic ring.

3. A pharmaceutical composition comprising an effective amount of a compound of formula (I) according to claim 1, or a salt thereof, together with one or more pharmaceutically acceptable adjuvants, excipients or diluents for use in enhancing image contrast in in vivo imaging or for treatment of a disease.

4. A pharmaceutical composition comprising an effective amount of a compound of formula (II) according to claim 2, or a salt thereof, together with one or more pharmaceutically acceptable adjuvants, excipients or diluents for use in enhancing image contrast in in vivo imaging or for treatment of a disease.

5. (canceled)

6. A method of generating images of a human or animal body involving administering a compound of formula I according to claim 1 to said body, and generating an image of at least a part of said body to which said contrast agent has distributed.

7. A method of generating enhanced images of a human or animal body comprising a compound as claimed in claim 1, which method comprises generating an image of at least part of said body.

8. A method of monitoring the effect of treatment of a human or animal body with a drug to combat a condition associated with cancer, said method involving administering to said body a composition as claimed in claim 3 and detecting the uptake of said composition by cell receptors, said administration and detection optionally but preferably being effected repeatedly, e.g. before, during and after treatment with said composition.

9. A method of treating cancer or a related disease in a human or animal body which comprises the administration of an effective amount of a composition as claimed claim 3.

10. (canceled)

11. A method of generating images of a human or animal body involving administering a compound of formula II according to claim 2 to said body, and generating an image of at least a part of said body to which said contrast agent has distributed.

12. A method of monitoring the effect of treatment of a human or animal body with a drug to combat a condition associated with cancer, said method involving administering to said body a composition as claimed in claim 4 and detecting the uptake of said composition by cell receptors, said administration and detection optionally but preferably being effected repeatedly, e.g. before, during and after treatment with said composition.

13. A method of treating cancer or a related disease in a human or animal body which comprises the administration of an effective amount of a composition as claimed claim 4.
